(19) United States
(12) Patent Application Publication
(10) Pub. No.: US 2010/0304397 A1
(43) Pub. Date: Dec. 2, 2010

(54) Chromogenic test kit for detecting health conditions in saliva

(76) Inventors: Morgan C. Burns, Philadelphia, PA (US); Christopher Burns, Ipswich, MA (US)

Correspondence Address: Christopher Burns, 28 Skytop Road, Ipswich, MA, 01938, US

(21) Appl. No.: 12/455,359

(22) Filed: Jun. 2, 2009

(51) Int. Cl. G01N 33/53; C12M 1/34

(52) U.S. Cl. 435/7.1; 435/288.1

(57) ABSTRACT

A device and method for detecting diseases, disorders and health conditions in saliva or other body fluid. The method employs solid phase immunoassay and similar detecting processes along with one of several bioluminescent reactions such that the presence of specific biomarkers is reported visually on a chromogenic panel incorporated directly into the test kit. The device does not require electricity or refrigeration, and results in a small, sealed diagnostic packet that can be safely discarded or stored as necessary.

### CROSS REFERENCE

None

### FEDERALLY SPONSORED RESEARCH

None

### SEQUENCE LISTING OR PROGRAM

None

### BACKGROUND OF THE INVENTION

1. Field of Invention

This invention relates to a device and method for the collection and analysis of human and animal saliva in order to diagnose diseases, disorders and health conditions.

2. Prior Art

### 1. Saliva is a Reliable Indicator of Disease, Disorders and Health Conditions

A number of methods have been developed for reliably identifying diseases, disorders and health conditions using normal lab analysis of subject saliva:

(a) Canadian Patent No. 2,558,666 shows a method for detecting biomarkers in saliva, employing high-density oligonucleotide microassay in a laboratory setting. In particular, the method addresses the detection of oral cavity and Oropharyngeal squamous cell carcinoma. U.S. Pat. No. 6,670,141 shows a method for detecting the presence of a panel of salivary biomarkers statistically indicative of breast cancer in women. The test is performed in a lab. U.S. Pat. No. 6,102,872 shows a method for determining the subject's blood glucose level—a primary indicator in the management of diabetes. The measurement is made by performing a chemical analysis of the glucose level in the subject's saliva. U.S. Pat. No. 5,914,271 shows a method for determining the fertile period in a female by monitoring the calcium and magnesium concentrations present in saliva during the three-to-five day period immediately prior to ovulation. Standard laboratory analysis of the saliva is implied.

(b) In one method, a normal laboratory analysis of the saliva in a reagent changes the color of the reagent: U.S. Pat. No. 5,858,796 shows a method for analyzing saliva in a reagent containing FE3+, chloride ions and multi-atom alcohol. The process indicates the presence of diabetes, disorders of the pancreas, initial stages of hypertonic disease, and hypertension by color change in the reagent itself. A reference chart of colors and the conditions they indicate is part of the method.

(c) In one method, the presence of an infectious disease is reliably detected through spectrophotometric or chemical analysis: U.S. Pat. No. 5,686,237 shows a method of detecting the presence of infectious and non-infectious agents from an analysis of biomarkers in human and animal saliva. The method uses incubation combined with analysis to determine exposure to pesticides and hazardous agents, and then compares the observed levels to baseline data for relevant controlled populations.

(d) At least one method uses an image of the crystal structure of dried saliva to detect the presence of a specific condition: U.S. Pat. No. 5,572,370 shows a method of detecting the fertile period for a woman by examining the crystal structure of saliva on a slide. The method employs equipment for depositing saliva on a slide, and then, after the saliva has dried and crystallized, magnifying the image and comparing it to known crystal structures that are indicative of fertility.

(e) Several methods have been developed to identify diseases by conjugating laboratory-developed antigens to biomarkers found in saliva that have a known correlation to the existence of those conditions: U.S. Pat. No. 5,695,930 shows a solid phase immunoassay method for detecting HIV antibodies in saliva. The method includes causing the HIV P17 protein antigen in saliva to conjugate with an antibody, which in turn conjugates to a reporter molecule, with the result that there is a color change in the liquid reactants. U.S. Pat. No. 5,792,605 shows a method for detecting the Hepatitis A virus in saliva with 99% sensitivity using an ELISA assay. Both patents require filtering and washing of the saliva sample using traditional laboratory equipment.

It is well known that human and animal saliva presents a rich supply of biomarkers from which diseases, disorders and other health conditions can be inferred. (Theime et al, 1992, Parry, et al, 1987, Kharchenko, 1992) Table 1 shows the diseases can be detected this way, along with references to supporting research.

TABLE 1
Diseases for which saliva has been shown to be a reliable indicator.

| | Affected (Deaths) | | | |
|---|---|---|---|---|
| Disease/Condition | Annual, Worldwide | | Notes | Reference |
| Avian Flu^{(v)} | 2 | Billion (100 M) | Pandemic likely in next 5 years. | 31, 32, 33 |
| Malaria^{(p)} | 500 | Million (2 M) | Rising again. Most victims are children | 1 |
| Hepatitis B^{(v)} | 350 | Million (103K) | Most common infectious disease. | 2, 3 |
| Depression^{(c)} | 240 | Million (400K) | Regulate medication. Most common mental illness. | 34, 35, 36 |
| Hepatitis C^{(v)} | 180 | Million (53K) | Most cases, even chronic, are asymptomatic. | 2, 3 |
| Diabetes^{(m)} | 171 | Million (3 M) | 18-20% of people over 60 are affected. | 4, 5, 6 |
| Schistosomiasis^{(p)} | 120 | Million (44K) | 20 million suffer severely from flukeworm. | 7, 8 |
| Dengue Fever^{(v)} | 50 | Million (150K) | Asia and Africa, mosquito borne, rarely fatal. | 9, 10, 11 |
| HIV/AIDS^{(v)} | 33 | Million (2.1 M) | Fatal, incurable, not transmitted thru saliva. | 12, 13, 14 |
| Measles^{(v)} | 30 | Million (500K) | Still prevalent in developing countries. | 15, 16 |
| Pneumonia^{(b/v)} | 20 | Million (4 M) | Leads to otitis media (6M) and meningitis | 29, 30 |
| Strep Throat^{(b)} | 15 | Million (3 M) | Symptoms often confused with flu. | 17, 18 |
| Tuberculosis^{(b)} | 15 | Million (2 M) | 1 billion infected in next 10 years (WHO) | 19, 20 |
| Typhoid^{(b)} | 15 | Million (600K) | Spread thru infected food, water. | 21 |
| Leishmaniasis^{(p)} | 12 | Million (51K) | Sandfly disease, infected people prone to relapse. | 22 |
| Influenza^{(v)} | 5 | Million (400K) | 20,000 deaths in US every year. | 31, 32, 33 |
| Rotavirus^{(v)} | 2 | Million (840K) | Kills 600,000 children/yr in developing countries. | 23, 24 |
| Hepatitis A^{(v)} | 1.4 | Million | Highly contagious. | 2, 3 |
| Meningitis^{(b/v)} | 1.2 | Million (173K) | Worldwide. Bacterial is more lethal. | 25, 26 |

^{(v)}= virus,
^{(b)}= bacteria,
^{(p)}= parasite,
^{(c)}= chemical

### 2. Methods and Devices for Saliva Analysis

Although this research continues to demonstrate the reliability of saliva as an indicator of disease, disorders and health conditions, most current methods employ laboratory procedures which would be impossible to replicate in the field or in the privacy of the home. (i) Access to saliva is not always easy. Patients may be unable to produce saliva, or for personal reasons may be unwilling or unable to spit. (ii) The volume of saliva gathered may not be sufficient to support the traditional laboratory analysis, or the analyte may be too diluted to be detected. (iii) Saliva may contain particulates, large molecules, and proteins that impede collection and interfere with analysis. (iv) The saliva itself may be contagious and present a danger to others, including the health workers. This is particularly true in the case of the H5N1 virus. (v) Most processes used for analyzing saliva involve incubation over several days under controlled temperatures, and employ centrifuges, microscopes, gas chromatography equipment and microprocessors. Current saliva diagnostic procedures usually require that the sample be flown to a laboratory, with the associated risk of loss of confidentiality, sample degradation and mix-up. Results are often not available for days.

As a result, in spite of its promise as a diagnostic medium, saliva is difficult to analyze in those settings where immediate diagnosis is most crucial, such as in a rural health survey, an epidemic, a medical emergency, or in the midst of a chemical or biological attack. In the case of contagious diseases, for example, the opportunity to identify, quarantine and treat the infectious person or animal immediately is lost because of the days consumed in laboratory processing. In the case of routine health maintenance functions such as testing for blood sugar, pregnancy, HIV/AIDS, or medication levels, saliva offers a reliable, real time, non-invasive indicator, but the equipment and skills required to complete the prevalent analysis are beyond the reach of the normal person.

Many attempts have been made to address these issues: (a) A number of methods and devices address the process of swabbing the mouth with a sponge or pad and then extracting the saliva: U.S. Pat. No. 5,714,341 shows how the saliva sample can be put in contact with a chromogenic substrate to determine sample sufficiency. U.S. Pat. No. 4,817,632 shows how a saliva swab or sponge can be enveloped in a porous membrane to exclude particulates and large molecules of no diagnostic interest. U.S. Pat. No. 5,981,300 shows how the swab can be treated with a pH measurement agent so that it will change color immediately when the patient being tested is at risk for tooth decay. U.S. Pat. No. 5,103,836 shows that the swab or sponge can be made to absorb more saliva by treating it in advance with a hypertonic solution. U.S. Pat. No. 4,418,702 shows how saliva can be absorbed by a swab and then squeezed onto a slide using a barrel-piston device.

(b) U.S. Pat. No. 6,022,326 describes a method of collection in which the patient aspirates into a tube and the saliva is collected from a special chamber.

(c) U.S. Pat. No. 5,935,864 describes a device by which saliva is drawn by capillary action into an analysis chamber where it is exposed to test strips that will indicate the potential for tooth decay.

(d) U.S. Pat. No. 4,774,962 describes a chewable material which collects the patient's saliva. The saliva is then extracted from the chewable material by centrifuge. U.S. Pat. No. 5,910,122 describes a nipple-shaped device which is chewed by the patient such that the saliva is collected into an attached chamber for subsequent laboratory analysis.

(e) U.S. Pat. No. 6,960,179 describes a portable device for examining the crystal structure of the saliva in order to determine the time of ovulation. U.S. Pat. No. 5,572,370 shows how microprocessor-based image analysis software can be used to similarly evaluate the crystal structure in saliva.

(f) U.S. Pat. No. 6,061,586 shows how electrolysis can be applied to saliva and the results analyzed by a microprocessor. The method is specifically intended to measure the level of lithium and other chemical components normally found in psychotropic drugs.

(g) U.S. Pat. No. 5,858,796 shows how a saliva sample can be quickly assayed in a solution of iron or chloride ions, causing it to turn color, indicating diabetes, disorders of the pancreas, initial stages of hypertonic disease, diabetes SD2, or hypertension.

None of these devices and methods for analyzing saliva address the problem of timeliness. In the case of the Avian flu, infected persons are contagious for several days before exhibiting any symptoms, even though a test of their saliva would reliably indicate their condition. This means that infected persons will remain undiagnosed, able to travel freely and infect others. It also means that antivirals, which could be very effective at the onset of the disease, may be too late by the time the normal diagnosis is confirmed.

Nor do the solutions already proposed address the problem of handling contagious material. In the case of a flu virus, and particularly in the case of H5N1, the primary means of human-to-human contagion is the distribution of saliva and expectorate. Most of the current procedures for collecting and analyzing saliva involve transportation and handling of the saliva by multiple health workers, using expensive laboratory analysis that would not normally be possible in the poor and densely populated areas where infections like HIV/AIDS, Hepatitis, Tuberculosis and the flu spread most rapidly.

While solutions have been offered that use the immunoassay process to bind antigens to biomarkers of various diseases, they still rely on time consuming laboratory processes to determine the results of the diagnosis. None, to our knowledge, offer the further step of binding the antigen/biomarker molecule to reporting molecules on a chromogenic panel so that the results of the assay can be seen quickly, without electricity, refrigeration or laboratory equipment. This improvement is crucial in identifying and isolating infected individuals in a rural setting, and in containing flu-like diseases which do not exhibit symptoms in their early, highly infectious stages.

### 3. Chromogenic Processes Employed in the Detection of Disease

Several methods have been developed that use the resonance energy transfer reaction to detect the presence of specific animal proteins in living animals. Such chromogenic processes, per se, are not the subject of this invention—which is a device and method for accommodating a wide range of biochemical reactions which cause an indicator medium to change in color or luminescence. But these prior inventions show that chromogenic processes in general, and resonance energy transfer in particular, have become well known laboratory tools.

(a) U.S. Patent Application No. 20070077609 describes a chain of proteins and antigens that effectively capture energy produced as a function of protein/protein interaction, and discharge that energy in the form of light. This patent describes the use of the Renilla luciferase with a fluorescent protein to determine protein interactions.

(b) U.S. Patent Application No. 20080057497, describes a method for capturing and amplifying the bioluminescence of certain resonance energy transfer reactions in order to enable their detection by electronic scanners and CCD arrays. This invention describes antibodies conjugated to antibodies and using those antibodies to detect levels of antigen of DNA in a sample. It also describes using differently colored labels to detect multiple antigens in the same sample.

(c) U.S. Pat. No. 5,518,887, describes a laboratory method for measuring the presence of an analyte by using antibodies. In some cases the reaction is measured by the extent to which the incubated sample changes color.

(d) U.S. Pat. No. 4,824,784, describes the use of a chromogenic agent, in combination with enzymes and antibodies, to measure the presence of a particular antibody such as might result from a disease, disorder or health condition. This patent expired Nov. 9, 2007.

While each of these patents extends the chromogenic tools available in a laboratory, none incorporate a device and method for performing such chromogenic diagnostics in the field where the detection of disease could have the greatest impact.

### SUMMARY

The invention described here includes a device and method for completing an analysis of filtered, size-selected saliva, and for presenting the results of that analysis minutes later in a simple, visual manner.

The device is a test kit for field and home use which includes (a) a reservoir for the collection of saliva; (b) one or more analytic sponges containing the chemistry by which the analysis is performed, and (c) a chromogenic panel attached to each sponge which reports the results of the analysis in visual form.

The method of the invention is (a) gather a sample of saliva into the reservoir. (b) Fold the sponges into the reservoir. When the saliva comes in contact with the antigens in the sponge or sponges, the analysis will cause the biomarkers for which the test kit has been configured to become attached to the chromogenic panel. (c) When the reaction is complete, a visible pattern will appear on the surface of the chromogenic panel indicating the presence of the biomarker in the saliva—a virus, a protein, bacteria, metabolites or chemicals which alone or together indicate the presence of the disease, disorder or condition.

The advantages of the device and method described here are (a) the device is simpler to use and less expensive than the laboratory analysis equipment customarily relied upon for saliva analysis, and as a result it can be widely deployed in rural areas and epidemic situations. (b) The chromogenic panel and the method of binding the antigen/biomarker molecules to reporting molecules on the panel make it possible to see the results of the analysis on site, within minutes.

### DRAWINGS—REFERENCE NUMERALS

10—Test kit cover

11—Test kit support

12—Reservoir

13—Transparent sheet

14—Strip of sealing adhesive

15—Chromogenic panel

16—Analytic sponge

17—Size excluding membrane

18—Biomarker antigen

19—Saliva

20—Absorbent tissue

21—Particulate

22—Saliva antigen

23—Biomarker

24—Biomarker and biomarker antigen

25—Saliva antigen and reporter molecule

26—Saliva reporting molecule—luminescent

27—Biomarker reporting molecule—luminescent

### DRAWINGS

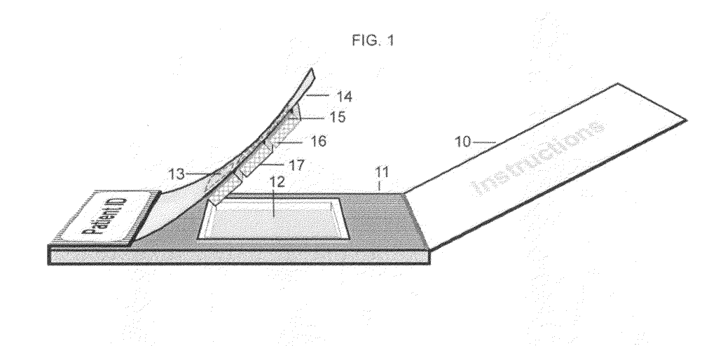

FIG. 1 is an overview of the invention in its preferred embodiment, showing the basic elements of the device before the saliva is deposited in the reservoir.

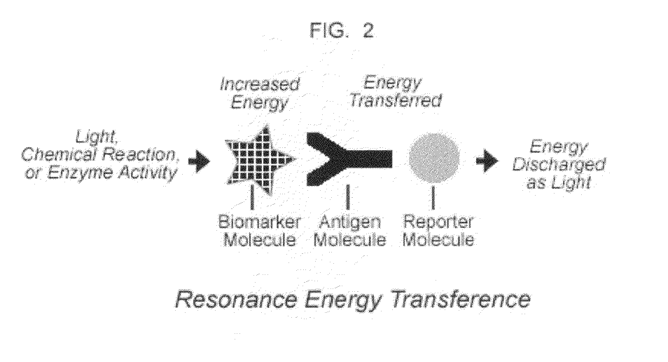

FIG. 2 is an illustration of the resonance energy transference process by which the energy generated during the conjugation of the biomarker and the biomarker antigen is transferred to the reporter molecule and then discharged as visible light.

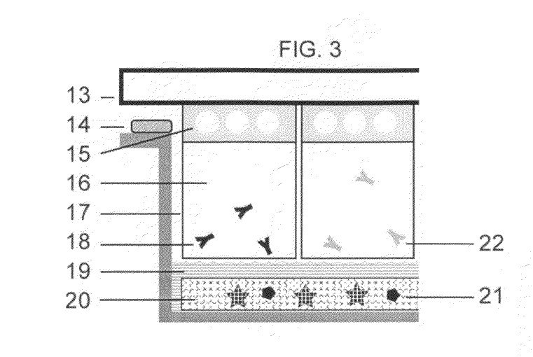

FIG. 3 is a cross section of the sponge and the reservoir before the diagnostic analysis begins. The saliva sample (19) or the absorbent tissue containing the saliva (20) is placed in the reservoir. The analytic sponges (16), each with a chromogenic panel (15) and encased in a size-excluding membrane (17) are lowered into the reservoir and the transparent sheet (13) is sealed to the lip of the reservoir with the exposed sealing strip (14).

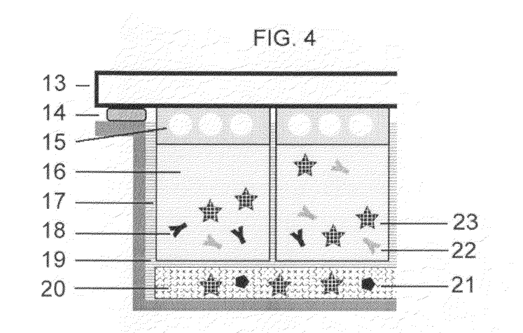

FIG. 4 is a cross section of the sponge and the reservoir during the second stage of the process. The action of sealing the reservoir pushes the saliva (19) up through the membrane (17) into each sponge (16), excluding large molecules and particulates (21). The sponge configured to detect the presence of the disease, disorder or health condition is imbued with antigens (18) designed to bind with a particular biomarker (23), usually a protein molecule indicative of a particular infection, disease or health condition. The sponge designed to detect saliva is imbued with antigens (22) designed to react in the presence saliva.

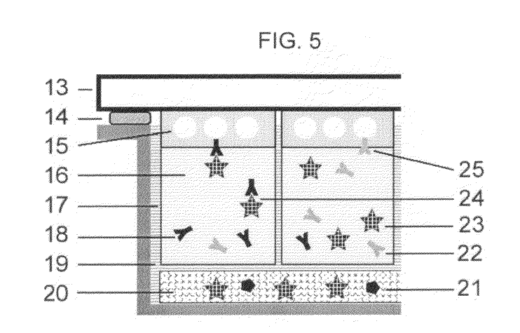

FIG. 5 is a cross section of the reservoir and sponges in the third stage of the process when the antigens bind to their target molecules. In the biomarker sponge, the F_{AB} end of the biomarker antigen becomes attached to the biomarker molecule (24). In the sponge configured to test for saliva, the saliva antigen (22) binds to molecules characteristic of saliva.

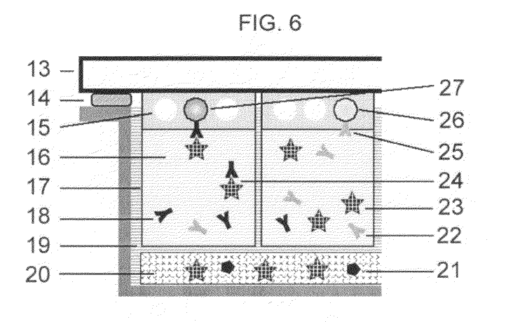

FIG. 6 is a cross section of the reservoir and sponges in the final stage of the process when the antigens in both the saliva and the biomarker sponges become attached to the chromogenic layer and trigger the resonance energy transfer process. In the biomarker sponge, the F_{C} end of the biomarker antigen binds to the reporter molecule on the chromogenic panel. (15) In the saliva sponge, the saliva antigen binds to the saliva reporting molecule on the chromogenic panel. (15) The heightened level of energy created by light, by an enzyme action, or by the particular chemistry of the sponge is now transferred from the antigen to the reporter molecule, which discharges the energy in the form of visible light. (26, 27)

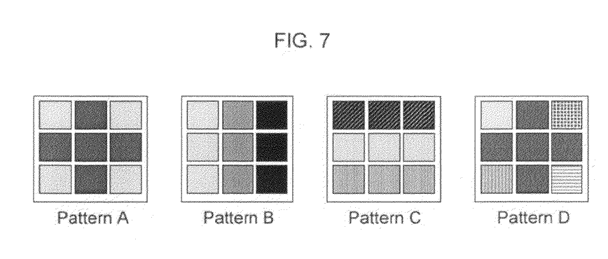

FIG. 7 shows how the color of the sponge or sponges can be used to indicate a number of diagnoses. In a nine-sponge matrix, for example, sponges may be designed to report in Pattern A where the central sponges in the array test for a single biomarker while the corner sp_{ong}es test only for the presence of saliva, and provide a confirming indication that the test is complete, even when the results are negative.

Some sponges may test for particular levels of concentration. In Pattern B, sponges with a lower affinity of the antigen for the biomarker produce a lower level of reaction and therefore present fainter colors.

In Pattern C, three sets of sponges may test for three different conditions, and in Pattern D a test for a single condition may be supported by four different tests for co-occurring conditions that support the diagnosis. In combination, this array of sponges gives the test kit a degree of redundancy and sensitivity as well as range.

### DETAILED DESCRIPTION—PREFERRED EMBODIMENT

In the preferred embodiment the device is a simple, disposable packet (FIG. 1) including a plastic reservoir 1 centimeter deep and 4 centimeters square (12) set in a cardboard and foam core block (11) with a matchbook cover (10). A transparent sheet (13) is bound into the kit to which is attached a group of nine analytic sponges (16), each integrated with a transparent chromogenic panel (15), and all encased in a size-excluding membrane (17) to keep out large molecules and particulate. Before use, the sponges are protected from the air by a sheet of impermeable foil that is removed and discarded at the time of the test. Removing the protective foil also exposes a sealing strip of adhesive (14) around the perimeter of the sponges. When the test is complete, the results are visible through the transparent sheet.

A separate saliva absorbing tissue included in the kit is made of a size-excluding polysaccharide matrix embedded with pilocarpine and a saliva indicator. The crystallized pilocarpine dissolves on the tongue and stimulates salivation. The saliva is absorbed by the fibers of the tissue while particles of a certain size are excluded. When the saliva reacts with the saliva indicator, the color of the tissue changes to indicate that a sufficient sample of saliva has been absorbed.

Each of the nine sponges is one centimeter square and about 0.5 centimeters thick when fully saturated with saliva. (FIG. 2) Each sponge is encased in a size-excluding membrane designed to permit molecules the size of the biomarker or smaller, while excluding larger molecules and particulates. The top of each sponge is attached to a chromogenic panel—a thin film that in turn is attached to the transparent sheet.

In manufacturing and distributing the device, test kits are configured to detect a specific disease, disorder or health condition. Detecting chemistry is chosen from many available chromogenic processes, some patented, and the analytic sponges are prepared with antigens and reporting molecules appropriate to the detecting task. For example, a flu version of the device might be configured with chemistry and reporting molecules appropriate to detecting the protein antibodies present in the saliva of a person with the flu. Another version of the kit may be configured with very different chemistry to detect the presence of metabolites indicating high blood sugar. The specific detecting chemistry and design of the reporting molecules are not within the scope of this invention.

In a test kit designed to identify a single condition such as the flu, five center sponges perform redundant tests for the same biomarker, providing multiple positive readings. The four corner sponges test for the presence of saliva, thereby providing a control indicating that the test is complete. In a kit designed to test for three conditions, such as the three common childhood diseases, malaria, rotavirus and measles, each row provides redundant indication of the appropriate biomarkers. In testing for the level of blood sugar, lithium or alcohol, the sponges can be designed to report different levels of concentration. In some difficult to diagnose situations, a primary cross of five sponges may be supplemented by four sponges testing for co-indications that may support the diagnosis. In this way test kits can be developed for health surveys specific to a particular region, bioterrorism assessment, accident documentation (alcohol, controlled substances, psychotropic drugs), and epidemic controls.

The method employs a class of chromogenic reactions that may vary in their specific chemistry, but which have in common the general behavior that when an antigen binds to its target—a protein molecule or a metabolite, for example—the resulting molecule discharges the excess energy in the form of light. Resonance energy transference (FIG. 2), as it is called, may be stimulated by infrared light (the Forster Resonance Energy Transfer—FRET), by a chemical reaction (CRET), or by an enzyme reaction (BRET). The chromogenic process chosen for each kit may vary depending on which disease, disorder or health condition the kit has been configured to detect.

Cross reactions are prevented by the design of the kit itself. The reporting molecules in each sponge are configured to bind to only one antigen, and when stimulated they emit only one color. Other antigens may flow into the sponge from neighboring sponges in the array, but they trigger no reaction since they cannot bind to the reporting molecules. In many chromogenic reactions, the chemistry must be “washed” between antigens to eliminate the cross reaction problem. But the segmented sponge design of the invention makes this washing step unnecessary.

### DETAILED DESCRIPTION—ALTERNATIVE EMBODIMENTS

(a) In an alternative embodiment intended for more advanced use in field stations, hospitals, laboratories, and other professionally staffed facilities, the invention uses a more complex chemistry in which the sponge or sponges are imbued with antibodies capable of binding to multiple biomarkers in the same saliva sample. The chromogenic display is therefore more complex and may include fluorescent reactions that are more difficult for the naked eye to detect. In those cases, an image acquisition device such as a CCD array is used to capture the results on the chromogenic panel, sometimes under special lighting conditions, and transmit those results to a computer processor. There the particular chromogenic patterns may be analyzed and interpreted, not only as a single observation but also in the context of other observations from the same demographic or geographic group.

(b) In another alternative embodiment intended for home use and health condition monitoring, the analytic sponge and sterile reservoir may be used to collect the saliva, supported by a stronger reusable container. The reservoir and sponges are then disposed of after completion of the test, while the container is kept for re-use.

Extensive research in recent years has shown that biomarkers in saliva are a reliable indicator of many diseases, disorders and health conditions. But the traditional methods for gathering and analyzing saliva have made it difficult to deploy this powerful diagnostic tool in rural areas where infectious diseases are particularly lethal and widespread. The device and method described here address this problem in a novel way by incorporating solid phase immunoassay and similar biomarker detection strategies along with a class of chromogenic reactions such as Fluorescence Resonance Energy Transfer (FRET), Chemiluminescence Resonance Energy Transfer (CRET), and Bioluminescence Resonance Energy Transfer (BRET). This combination of biotechnology tools are further embodied in a novel device which simplifies the process of gathering and analyzing saliva. Crucial to the utility of the device is that in its nominal embodiment it permits saliva analysis to be performed in minutes without electricity or refrigeration, delivering reliable diagnostic results in a rural village, at an airport or at a border checkpoint while the possibly infected person is within reach of immediate treatment or isolation. Alternative embodiments of this method and device include test kits for a wide range of health conditions including infection, substance abuse or other conditions of concern at home and in hospitals, schools and places of employment.

### REFERENCES

[1] Mharakurwa S, Simoloka C, Thuma P E, Shiff C J, Sullivan D J. “PCR detection of Plasmodium falciparum in human urine and saliva samples.” Malar J. 2006 Nov. 8; 5:103.

[2] Amado L A, Villar L M, de Paula V S, de Almeida A J, Gaspar A M. “Detection of hepatitis A, B, and C virus-specific antibodies using oral fluid for epidemiological studies.” Mem Inst Oswaldo Cruz. 2006 March; 101(2):149-55.

[3] Mackiewicz V, Dussaix E, Le Petitcorps M F, Roque-Afonso A M. “Detection of hepatitis A virus RNA in saliva.” J Clin Microbiol. 2004 September; 42(9):4329-31.

[4] Toda M, Tsukinoki R, Morimoto K. “Measurement of salivary adiponectin levels.” Acta Diabetol. 2007 March; 44(1):20-2.

[5] Aydin S. “A comparison of ghrelin, glucose, alpha-amylase and protein levels in saliva from diabetics.” J Biochem Mol Biol. 2007 Jan. 31; 40(1):29-35.

[6] Todd A L, Ng W Y, Lee Y S, Loke K Y, Thai A C. “Evidence of autoantibodies to glutamic acid decarboxylase in oral fluid of type 1 diabetic patients.” Diabetes Res Clin Pract. 2002 September; 57(3):171-7.

[7] Wang Z, Xue C, Lou W, Zhang X, Zhang E, Wu W, Shen G. “Non-invasive immunodiagnosis of Schistosomiasis japonica: the detection of specific antibodies in saliva.” Chin Med J (Engl). 2002 October; 115(10):1460-4.

[8] Santos M M, Garcia T C, Orsini M, Disch J, Katz N, Rabello A. “Oral fluids for the immunodiagnosis of Schistosoma mansoni infection.” Trans R Soc Trop Med Hyg. 2000 May-June;94(3):289-92.

[9] Chakravarti A, Matlani M, Jain M. “Immunodiagnosis of dengue virus infection using saliva.” Curr Microbiol. 2007 December; 55(6):461-

[10] Mizuno Y, Kotaki A, Harada F, Tajima S, Kurane I, Takasaki T. “Confirmation of dengue virus infection by detection of dengue virus type 1 genome in urine and saliva but not in plasma.” Trans R Soc Trop Med Hyg. 2007 July; 101(7):738-9.

[11] Balmaseda A, Guzmán M G, Hammond S, Robleto G, Flores C, Téllez Y, Videa E, Saborio S, Pérez L, Sandoval E, Rodriguez Y, Harris E. “Diagnosis of dengue virus infection by detection of specific immunoglobulin M (IgM) and IgA antibodies in serum and saliva.” Clin Diagn Lab Immunol. 2003 March; 10(2):317-22.

[12] King S D, Wynter S H, Bain B C, Brown W A, Johnston J N, Delk A S. “Comparison of testing saliva and serum for detection of antibody to human immunodeficiency virus in Jamaica, West Indies.” J Clin Virol. 2000 December; 19(3):157-61.

[13] Spielberg F, Critchlow C, Vittinghoff E, Coletti A S, Sheppard H, Mayer K H, Metzgerg D, Judson F N, Buchbinder S, Chesney M, Gross M. “Home collection for frequent HIV testing: acceptability of oral fluids, dried blood spots and telephone results.” HIV Early Detection Study Group. AIDS. 2000 Aug. 18; 14(12):1819-28.

[14] Martinez P M, Torres A R, Ortiz de Lejarazu R, Montoya A, Martin J F, Eiros J M. “Human immunodeficiency virus antibody testing by enzyme-linked fluorescent and westernblot assays using serum, gingival-crevicular transudate, and urine samples.” J Clin Microbiol. 1999 April; 37(4):1100-6.

[15] Mokhtari Azad T, Ehteda A, Yavari P, Hamkar R, Safar Pour Z, Essalat M, Nategh R. “Comparative Detection of Measles Specific IgM Antibody in Serum and Saliva by an Antibody-Capture IgM Enzyme Immunoassay (EIA).” Iran J Allergy Asthma Immunol. 2003 September; 2(3):149-54.

[16] Chibo D, Riddell M A, Catton M G, Birch C J. “Applicability of oral fluid collected onto filter paper for detection and genetic characterization of measles virus strains.” J Clin Microbiol. 2005 July; 43(7):3145-9.

[17] Kumagai K, Sugano N, Takane M, Iwasaki H, Tanaka H, Yoshinuma N, Suzuki K, Ito K. ‘Detection of Streptococcus anginosus from saliva by real-time polymerase chain reaction.” Lett Appl Microbiol. 2003; 37(5):370-3.

[18] De Soet J J, De Graaff J. “Monoclonal antibodies for enumeration and identification of mutans streptococci in epidemiological studies.” Arch Oral Biol. 1990; 35 Supp1:165S-168S.

[19] Wilson S M, Nava E, Morales A, Godfrey-Faussett P, Gillespie S, Andersson N., ‘Simplification of the polymerase chain reaction for detection of Mycobacterium tuberculosis in the tropics.” Trans R Soc Trop Med Hyg. 1993 March-April; 87(2):177-80.

[20] Del Pezzo M, Alifano M, Faraone 5, Battiloro R, De Pascalis R, Lavitola A., “Detection of IgA against the mycobacterial antigen A60 in serum and saliva in patients with active pulmonary tuberculosis: preliminary results.” New Microbiol. 1996 October; 19(4):363-7.

[21] Herath H M. “Early diagnosis of typhoid fever by the detection of salivary IgA.”J Clin Pathol. 2003 September; 56(9):694-8.

[22] Rohousova I, Ozensoy S, Ozbel Y, Volf P. “Detection of species-specific antibody response of humans and mice bitten by sand flies.” Parasitology. 2005 May; 130(Pt 5):493-9.

[23] Ward R L, Pax K A, Sherwood J R, Young E C, Schiff G M, Bernstein D I. “Salivary antibody titers in adults challenged with a human rotavirus.”J Med Virol. 1992 March; 36(3):222-5.

[24] Grimwood K, Lund J C, Coulson B S, Hudson I L, Bishop R F, Barnes G L. “Comparison of serum and mucosal antibody responses following severe acute rotavirus gastroenteritis in young children.” J Clin Microbiol. 1988 April; 26(4):732-8.

[25] Görögh T, Rudolph P, Meyer J E, Werner J A, Lippert B M, Maune S. “Separation of beta2-transferrin by denaturing gel electrophoresis to detect cerebrospinal fluid in ear and nasal fluids.” Clin Chem. 2005 September; 51(9):1704-10.

[26] Thys J P, Jacobs F, Byl B. “Microbiological specimen collection in the emergency room.” Eur J Emerg Med. 1994 March; 1(1):47-53.

[27] Lejon V, Jamonneau V, Solano P, Atchade P, Mumba D, Nkoy N, Bébronne N, Kibonja T, Balharbi F, Wierckx A, Boelaert M, Büscher P. “Detection of trypanosome-specific antibodies in saliva, towards non-invasive serological diagnosis of sleeping sickness.” Trop Med Int Health. 2006 May; 11(5):620-7.

[28] Lejon V, Kwete J, Büscher P. “Towards saliva-based screening for sleeping sickness?” Trop Med Int Health. 2003 July; 8(7):585-8.

[29] Foo R L, Graham S M, Suthisarnsuntorn U, Parry C M. “Detection of pneumococcal capsular antigen in saliva of children with pneumonia.” Ann Trop Paediatr. 2000 June; 20(2):161-3.

[30] Perlino C A, Shulman J A. “Detection of pneumococcal polysaccharide in the sputum of patients with pneumococcal pneumonia by counterimmunoelectrophoresis.”J Lab Clin Med. 1976

[31] Harold C. Slavkin Toward Molecularly Based Diagnostics For The Oral Cavity. JADA, Vol 129, August 1998

[32] D. Malamud H. Bau S. Niedbala P. Corstjens Point Detection of Pathogens in Oral Samples Adv Dent Res 18:12-16 June 2005

[33] Bourinbaiar A S, Timofeev I V, Agwale S M. Recent advances in development of avian flu and influenza diagnostics. Expert Rev Mol Diagn. 2006 November;6(6):783-95.

[34] Saeeduddin Ahmed, P. David Mozley, and William Z. Potter, Biomarkers in Psychotropic Drug Development Am J Geriatr Psychiatry 10:678-686, December 2002

[35] G. Lac Saliva assays in clinical and research biology Pathologie Biologie, Volume 49, Issue 8, 2001, Pages 660-667

[36] Phillip. Gold New insights into the role of cortisol and the glucocorticoid receptor in severe depression. Biological Psychiatry, Volume 52, Issue 5, Pages 381-385

[37] Parry et al, “Sensitive assays for viral antibodies in saliva, an alternative to tests on serum,” Lancet Jul. 11, 1987 pp. 72-75

[38] Kharchenko et al, “New Approches to Diagnosing . . . ” Izv.Akad.Nowk (Russian). Ser Biol. 1992, No 4, pp. 575-581.

[39] Macy et al, “Enhanced ELISA, how to measure less than 10 picograms of a specific protein (immunoglobulin) in less than 8 hours,” The FASEB Journal, Vol 2, pp 3003-3009, November 1988.

[40] T. Thieme, P. Yoshihara, S. Piacentini and M. Beller, “Clinical evaluation of oral fluid samples for diagnosis of viral hepatitis,” J Clin Microbiol. 1992 May; 30(5): 1076-1079.

[41] Granfors, Kaisa, “Measurement of Immunoglobulin M (IgM), IgG, and IgA Antibodies Against Yersinia enterocolitica by Enzyme-Linked Immunosorbent Assay: Persistence of Seruin Antibodies During Disease,” Journal of Clinical Microbiology, Vol 9, No 3 (March, 1979) pp 336-341.

[42] Herr, Amy et al., “Microfluidic immunoassays as rapid saliva-based clinical diagnostics,” Proceedings of the National Academy of Sciences, 104 (2007) pp 5268-5273.

[43] Sreebny, Leo M., “Saliva in health and disease: an appraisal and update,” International Dental Journal (2000) 50, 140-161

[44] Haeckel R, et al, “The Application of saliva, sweat and tear fluid for diagnostic purposes,” Ann de Biol Clin (1993) 51: pp 903-910.

[45] Siegel, I. A et al, “The role of saliva in drug monitoring. Saliva as a Diagnostic Fluid.” Ann New York Acad Sci 1993 694: 86-90.

[46] Streckfus, C. F., Bigler L. R., “Saliva as a diagnostic fluid,” Oral Dis 8(2) 69-76.

[47] I. D. Mandel, “The diagnostic uses of saliva,” J. Oral Pathol. and Medicine 19:119-125 (1990).

[48] “SALIgAE® For The Forensic Identification of Saliva, Technical Information Sheet,” Abacus Diagnostics, Inc.

[49] Auvdel, M. J., “Amylase Levels in Semen and Saliva Stains,” Journal of Forensic Sciences, JFSCA, Vol. 31, No. 2, April 1986, pp. 426-431.

[50] Gaensslen, R. E., “Section 11. Identification of Saliva,” Sourcebook in Forensic Serology, Immunology, and Biochemistry, NIJ, 1983, pp. 183-189.

[51] Willot, G. M. “An Improved Test for the Detection of Salivary Amylase in Stains,” Journal of The Forensic Science Society, JFSS (1974), 14, 341.

[52] Keating, S. M. and Higgs, D. F. “The detection of amylase on swabs from sexual assault cases,” Journal of The Forensic Science Society, JFSS, 1994; 34(2):89-93.

[53] Miller, D. W. and Hodges, J. C., “Validation of Abacus SALIgAE® Test for the Forensic Identification of Saliva,” West Virginia State Police. p. 1-19 (2005).

[54] Silenieks, E., “SALIgAE® Test: The Detection of Salivary Amylase in Expirated Blood Patterns Technical Note,” p. 1-6 2005).

[55] Merritt A D, Rivas M L, Bixler D, Newell R., “Salivary and pancreatic amylase: electrophoretic characterizations and genetic studies,” Am J Hum Genet 1973; 25:513-22.

[56] Whitehead P H and Kipps A E., “The significance of amylase in forensic investigations of body fluids,” Journal of Forensic Sciences 6(3): p 137-44.

We claim:

1. A device for analyzing saliva and other body fluids in order to detect the presence of diseases, disorders and other specific health conditions, comprising:
(a) a container designed and constructed of a material suitable to support, hold and preserve a reservoir, an analytic sponge, and a chromogenic panel,
(b) said reservoir for collecting fluid, large enough to hold said analytic sponge as well as a sample of body fluid sufficient to complete successfully the detecting chemistry,
(c) said analytic sponge made of absorbent beads or material and containing one of a plurality of said detecting chemistries designed to react with specific biomarkers in such a way as to cause a visible change in said chromogenic panel, and
(d) said chromogenic panel which changes visibly as a result of the analytic process,
whereby the presence of said disease, disorder or specific health condition can be quickly determined by visual inspection of said chromogenic panel.

2. A size selecting membrane encloses said analytic sponge in claim 1, filtering out unwanted particulate as well as molecules larger than the biomarker of interest.

3. Said chromogenic panel in claim 1 is affixed to said analytic sponge in such a way that the reaction between the detecting chemistry and the biomarkers occurring in said body fluid causes the surface of said chromogenic panel to visibly change.

4. Said chromogenic panel in claim 1 is divided into one or more reporting sections, each prepared to react to one of a plurality of detected conditions.

5. Said chromogenic panel in claim 1 contains at least one area which visibly reacts to the presence of normal body fluid alone.

6. Said chromogenic panel in claim 1 may visibly react to said detecting chemistry in a plurality of ways, including changing color, creating visible patterns, and producing light that is visible to the aided or unaided eye.

7. Said chromogenic panel in claim 1 may visibly react to said detecting chemistry in a manner that can be identified by a highly sensitive electronic scanner or CCD array.

8. Said container is so constructed that said body fluid, said analytic sponge, and said chromogenic panel become sealed together on completion of the test in a partially transparent packet, whereby the sample of body fluid being tested may not thereafter leak or become contaminated as a result of normal handling and storage.

9. A method for detecting the presence of specific diseases, disorders and health conditions from a sample of saliva or other body fluid, wherein:
(a) a reservoir collects a sufficient sample of said saliva or fluid,
(b) an analytic sponge imbued with a detecting chemistry specific to a particular disease, disorder or health condition is immersed in said fluid,
(c) said detecting chemistry reacts with the biomarkers in said fluid which are indicative of the disease, disorder or health condition which said test kit is configured to detect,
(d) said detecting chemistry changes as a result of contact with said biomarkers, and in turn causes a reaction in reporting molecules in the chromogenic panel,
(e) said reporting molecules change the appearance of said chromogenic panel, and
(f) the pattern and intensity of color appearing on said chromogenic panel indicates the presence, absence or density of said biomarkers specific to the disease, disorder or health condition for which said test kit is configured.

10. Said analytic sponge in claim 9 is imbued with one of a plurality of said detecting chemistries designed to react with one or more said biomarkers.

11. Said detecting chemistry in claim 9 may consist of antigens or molecules which become bound to said biomarkers in said fluid, forming new molecules which in turn become bound to reporter molecules in the chromogenic panel.

12. Said detecting chemistry in claim 9 consists of antigens or molecules which bind to proteins, metabolites, hormones, minerals and other biomarkers in said fluid which are indicative of diseases, disorders or specific health conditions including, but not limited to, a virus or bacterial infection, high blood sugar, drug or alcohol use, pregnancy or poisoning.

13. Said detecting chemistry in claim 9 may incorporate a process commonly called solid-phase immunoassay in which the antigen binds with the antigenic region of said biomarker.

14. Said detecting chemistry in claim 9 may also incorporate a process in which said antigen binds in turn to said reporting molecule on said chromogenic panel.

15. Said reporter molecules in claim 9 are incorporated into said chromogenic panel in such a way that when activated by said antigens or molecules bound to said biomarkers they cause the appearance of said chromogenic panel to change.

16. Said reporter molecules in claim 9 may be one of a plurality of chromogenic configurations that include, but are not limited to, fluorescent proteins, luminescent proteins, chromophores, fluorophores, and luminophores.

17. Said chromogenic panel in claim 9 is designed so that all or a portion of the surface may change color, may become darker or lighter, or may emit light, in response to the activation of said reporter molecules.

18. The pattern of shape, density and color appearing on said chromogenic panel in claim 9 indicates the presence of viral or bacterial infection, metabolic imbalance, or other disorders or health conditions, based on a non-invasive sample of saliva or body fluid, without laboratory equipment, in time to comfort, treat or quarantine the patient.
